(19) United States
(12) Patent Application Publication
(10) Pub. No.: US 2020/0138023 A1
(43) Pub. Date: May 7, 2020

(54) MICROBICIDAL COMPOSITION

(71) Applicant: DDP Specialty Electronic Materials US 8, LLC, Wilmington, DE (US)

(72) Inventors: Megan Diehl, Wilmington, DE (US); Kiran Pareek, Wilmington, DE (US); Hiteshkumar Dave, Wilmington, DE (US)

(21) Appl. No.: 16/621,764

(22) PCT Filed: May 2, 2018
(86) PCT No.: PCT/US2018/030617

§ 371 Date: Dec. 12, 2019

Related U.S. Application Data
(60) Provisional application No. 62/520,608, filed on Jun. 16, 2017.

(51) Int. Cl. A01N 37/36; A01N 37/02; A61Q 17/00; A61K 8/36

(52) U.S. Cl. CPC A01N 37/36; A61K 8/361; A61Q 17/005; A01N 37/02

(57) ABSTRACT

A synergistic microbicidal composition comprising lactic acid and caprylic acid.

This invention relates to microbicidal compositions containing lactic acid and caprylic acid.

In some cases, commercial microbicides cannot provide effective control of certain microorganisms, even at high use concentrations, due to weak activity against certain types of microorganisms, e.g., those resistant to some microbicides, or due to aggressive environmental conditions. Combinations of different microbicides are sometimes used to provide overall control of microorganisms in a particular end use environment. However, there is still a need for additional combinations of microbicides, or combinations of microbicides with formulation ingredients or raw materials, or combinations of multifunctional ingredients, having enhanced activity against various strains of microorganisms to provide effective control of such microorganisms. In addition, there is still a need for combinations containing lower levels of individual microbicides for environmental and economic benefit. U.S. Pat. No. 7,820,594 is an example of one such case where microbicides are combined to control the growth of a microbial population. In the cited reference, lactic acid and caprylic are disclosed, however, the cited reference does not pair the actives together over a ratio range to demonstrate a synergistic effect. The problem addressed by this invention is to provide such additional combinations that demonstrate a synergistic effect.

The present invention is directed to a synergistic microbicidal composition comprising lactic acid and caprylic acid.

The present invention is further directed to a method of using a synergistic microbicidal composition comprising lactic acid and caprylic acid by adding the composition into cosmetics and toiletries such as lotion, hair styling cream, paste, or gum, conditioner, shampoo, body wash, shower gel, liquid soap, sunscreen lotion and spray, tanning lotion, skin care lotion, one and two-part hair dye, permanent waving formulation, micellar water, cleanser, mask solutions and wet wipes; and household, industrial and institutional products such as soaps, laundry detergents, fabric softener, automatic dish washing detergents, polishes and cleaners.

As used herein, the following terms have the designated definitions, unless the context clearly indicates otherwise. The term “microorganism” includes, for example, fungi (such as yeast and mold), bacteria, and algae. The term “locus” refers to an industrial system or product, a personal care system or product, or a home care system or product subject to contamination by microorganisms. The term “compound” refers to a microbicide, a formulation ingredient, or a raw material. The following abbreviations are used throughout this specification: ppm=parts per million by weight (weight/weight), mL=milliliter, ATCC=American Type Culture Collection, and MIC=minimum inhibitory concentration. Unless otherwise specified, temperatures are in degrees centigrade (° C.), and references to percentages (%) are by weight. Amounts of organic microbicides are given on an active ingredient basis in ppm (w/w). Ratios are by weight and may be expressed as, for example, 1/400 or 1:400.

The compositions of the present invention unexpectedly have been found to provide enhanced antimicrobial efficacy at levels lower than what would be expected for a combination of the compounds based on their individual efficacy.

The synergistic microbicidal composition of the present invention comprises lactic acid and caprylic acid. Lactic acid is also known as 2-hydroxypropanoic acid and is (CAS No. 50-21-5). Caprylic acid is also known as octanoic acid and is (CAS No. 124-07-2). The weight ratio of the lactic acid to caprylic acid is from 2000:1 to 1:10.

The compounds in the composition of this invention may be used “as is” or may first be formulated with a solvent or a solid carrier. Suitable solvents include, for example, water; glycol ethers such as phenoxyethanol, phenoxypropanol, dipropyleneglycol phenylether; alkyl glycerin ethers such as ethylhexylglycerin, cyclohexylglycerin, hexyl glycerin, glyceryl lauryl ether; alcohols, such as, for example, methanol, ethanol, propanol, phenethyl alcohol; diols such as propanediol, butanediol, pentanediol, pentylenediol, hexanediol, octanediol, decanediol, dodecanediol; ketones, such as, for example, acetone and methyl ethyl ketone; esters, such as, for example, glyceryl caprylate/caprate, sorbitol caprylate; and mixtures thereof. It is preferred that the solvent is selected from water, glycols, glycol ethers, and mixtures thereof. Suitable solid carriers include, for example, cyclodextrin, silicas, diatomaceous earth, waxes, cellulosic materials, alkali and alkaline earth (e.g., sodium, magnesium, potassium) metal salts (e.g., chloride, nitrate, bromide, sulfate), and charcoal.

When a compound is formulated in a solvent, the formulation may optionally contain surfactants. When such formulations contain surfactants, they are generally in the form of emulsion concentrates, emulsions, microemulsion concentrates, or microemulsions. Emulsion concentrates form emulsions upon the addition of a sufficient amount of water. Microemulsion concentrates form microemulsions upon the addition of a sufficient amount of water. Such emulsion and microemulsion concentrates are generally well known in the art; it is preferred that such formulations are free of surfactants. U.S. Pat. No. 5,444,078 may be consulted for further general and specific details on the preparation of various microemulsions and microemulsion concentrates.

A compound also may be formulated in the form of a dispersion. The solvent component of the dispersion may be an organic solvent or water, preferably water. Such dispersions may contain adjuvants such as, for example, co-solvents, thickeners, anti-freeze agents, dispersants, fillers, pigments, surfactants, biodispersants, sulfosuccinates, terpenes, furanones, polycations, stabilizers, scale inhibitors, and anti-corrosion additives.

When each compound is first formulated with a solvent, the solvent used for the first component may be the same as or different from the solvent used to formulate the other component. Water is the preferred solvent for many biocide applications. It is preferred that the two solvents are miscible.

Those skilled in the art will recognize that the compounds of the present invention may be added to a locus sequentially, simultaneously, or may be combined before being added to the locus. In one embodiment of the invention, the first component and the second component are added to a locus simultaneously or sequentially. When the components are added simultaneously or sequentially, each may independently contain adjuvants, such as, for example, solvent, thickeners, anti-freeze agents, colorants, sequestrants (such as ethylenediamine-tetraacetic acid, ethylenediaminedi succinic acid, iminodisuccinic acid and salts thereof), dispersants, surfactants, biodispersants, sulfosuccinates, terpenes, furanones, polycations, stabilizers, scale inhibitors and anti-corrosion additives.

The compositions of the present invention can be used to inhibit the growth of microorganisms or higher forms of aquatic life (such as protozoans, invertebrates, bryozoans, dinoflagellates, crustaceans, mollusks, etc) by introducing a microbicidally effective amount of the compositions onto, into, or at a locus subject to microbial attack. Suitable loci include, for example: industrial process water; electrocoat deposition systems; cooling towers; air washers; gas scrubbers; mineral slurries; wastewater treatment; ornamental fountains; reverse osmosis filtration; ultrafiltration; ballast water; evaporative condensers; heat exchangers; pulp and paper processing fluids and additives; starch; plastics; emulsions; dispersions; paints; latices; coatings, such as varnishes; construction products, such as mastics, caulks, and sealants; construction adhesives, such as ceramic adhesives, carpet backing adhesives, and laminating adhesives; industrial or consumer adhesives; photographic chemicals; printing fluids; household and personal care products, such as, for example, bathroom and kitchen cleaners; cosmetics; lotions, moisturizers, toiletries; hair styling creams, pastes, or gums; conditioners, 2 in 1 conditioning shampoos, body wash/shower gels, liquid soaps, sunscreen lotions and sprays, tanning lotions, skin care lotions, one and two-part hair dyes, permanent waving formulations, soaps; detergents; cleaners; floor polishes; laundry rinse water; metalworking fluids; conveyor lubricants; hydraulic fluids; leather and leather products; textiles; textile products; wood and wood products, such as, for example, plywood, chipboard, flakeboard, laminated beams, oriented strandboard, hardboard, and particleboard; petroleum processing fluids; fuel; oilfield fluids, such as injection water, fracture fluids, and drilling muds; agriculture adjuvant preservation; surfactant preservation; medical devices; diagnostic reagent preservation; food preservation, such as plastic or paper food wrap; food, beverage, and industrial process pasteurizers; toilet bowls; recreational water; pools; and spas.

In one embodiment, the compositions of the present invention are used to inhibit the growth of microorganisms at a locus selected from one or more of a cosmetic, lotion, toiletry, hair styling cream, paste, or gum, conditioner, shampoo, body wash, shower gel, liquid soap, sunscreen lotion and spray, tanning lotion, skin care lotion, one and two-part hair dye, permanent waving formulation, micellar water, cleanser, mask solutions, wet wipe solutions, soap, liquid laundry detergents, fabric softener, liquid and solid dish wash, and cleaning solutions.

### Materials and Methods

The synergism of the combination of the present invention was demonstrated by testing a wide range of concentrations and ratios of the compounds.

One measure of synergism is the industrially accepted method described by Kull, F. C.; Eisman, P. C.; Sylwestrowicz, H. D. and Mayer, R. L., in Applied Microbiology 9:538-541 (1961), using the ratio determined by the formula:

Qₐ/Q_{A}+Q_{b}/Q_{B}=Synergy Index (“SI”)

wherein:
Q_{A}=concentration of compound A (first component) in ppm, acting alone, which produced an end point (MIC of Compound A).
Qₐ=concentration of compound A in ppm, in the mixture, which produced an end point.
Q_{B}=concentration of compound B (second component) in ppm, acting alone, which produced an end point (MIC of Compound B).
Q_{b}=concentration of compound B in ppm, in the mixture, which produced an end point.

When the sum of Qₐ/Q_{A} and Q_{b}/Q_{B} is greater than one, antagonism is indicated. When the sum is equal to one, additivity is indicated, and when less than one, synergy is demonstrated. The lower the SI, the greater is the synergy shown by that particular mixture. The minimum inhibitory concentration (MIC) of a microbicide is the lowest concentration tested under a specific set of conditions that prevents the growth of the tested microorganisms.

Synergy tests were conducted using standard microtiter plate assays with media designed for optimal growth of the test microorganism. Potato Dextrose Broth (PDB medium) was used for testing yeast and mold. In this method, a wide range of combinations of microbicides and other personal care raw materials was tested by conducting high resolution MIC assays in the presence of lactic acid. High resolution MICs were determined by adding varying amounts of microbicide to one column of a microtitre plate and doing subsequent ten-fold dilutions using an automated liquid handling system to obtain a series of closely spaced endpoints.

The synergy of the combinations of the present invention was determined against a yeast, Candida albicans (C. albicans—ATCC 10231) and a mold, Aspergillus brasiliensis (A. brasiliensis—ATCC 16404). The yeast and mold were used at concentrations of 5×10⁵ cells per mL. These microorganisms are representative of natural contaminants in many consumer and industrial applications. The plates were visually evaluated for microbial growth (turbidity) to determine the MIC after various incubation times at 25° C.

The test results for demonstration of synergy of the combinations of the present invention are shown below in Table 1. In this test, First Component (A) was lactic acid and the Second Component (B) was caprylic acid. Table 1 shows the specific combinations of lactic acid and caprylic acid; results against the microorganisms tested with incubation times; the end-point activity in ppm measured by the MIC for lactic acid alone (Q_{A}), for caprylic acid alone (Q_{B}), for lactic acid in the mixture (Qₐ) and for caprylic acid in the mixture (Q_{b}); the calculated SI value; and the range of synergistic ratios for each combination tested (lactic acid/caprylic acid or a/b).

In each of the comparisons, the effective synergistic ratio may vary among the microorganisms tested and the various combinations of components A and B. Data in the tables below include the range of ratios that were found to be synergistic. Not all data which were collected outside of the synergistic ranges are reported.

### Tables

First Component (A)=Lactic acid (LA)
Second Component (B)=Caprylic acid (CA)
Table 1: Aspergillus brasiliensis
ATCC 16404
Potato Dextrose Broth
Contact Time=7 days
Table 2: Candida albicans
ATCC 10231
Potato Dextrose Broth
Contact Time=48 hours

TABLE 1

| Ratio | Lactic Acid, ppm | Caprylic Acid, ppm | SI |
|---|---|---|---|
| | 30,000 | 0 | 1.00 |
| 200:1 | 20,000 | 100 | 0.68 |
| 133:1 | 20,000 | 150 | 0.68 |
| 100:1 | 20,000 | 200 | 0.69 |
| 80:1 | 20,000 | 250 | 0.69 |
| 67:1 | 20,000 | 300 | 0.70 |
| 50:1 | 20,000 | 400 | 0.71 |
| 40:1 | 20,000 | 500 | 0.72 |
| 20:1 | 20,000 | 1,000 | 0.77 |
| 13:1 | 20,000 | 1,499 | 0.82 |
| 10:1 | 20,000 | 1,999 | 0.87 |
| 67:1 | 10,000 | 150 | 0.35 |
| 50:1 | 10,000 | 200 | 0.35 |
| 40:1 | 10,000 | 250 | 0.36 |
| 33:1 | 10,000 | 300 | 0.36 |
| 25:1 | 10,000 | 400 | 0.37 |
| 20:1 | 10,000 | 500 | 0.38 |
| 10:1 | 10,000 | 1,000 | 0.43 |
| 7:1 | 10,000 | 1,499 | 0.48 |
| 5:1 | 10,000 | 1,999 | 0.53 |
| 4:1 | 10,000 | 2,499 | 0.58 |
| 3:1 | 10,000 | 3,001 | 0.63 |
| 2:1 | 10,000 | 5,000 | 0.83 |
| 40:1 | 8,000 | 200 | 0.29 |
| 32:1 | 8,000 | 250 | 0.29 |
| 20:1 | 8,000 | 400 | 0.31 |
| 8:1 | 8,000 | 1,000 | 0.37 |
| 4:1 | 8,000 | 1,999 | 0.47 |
| 2:1 | 8,000 | 4,002 | 0.67 |
| 6:1 | 6,000 | 1,000 | 0.30 |
| 3:1 | 6,000 | 1,999 | 0.40 |
| 2:1 | 6,000 | 3,001 | 0.50 |
| 4:1 | 4,000 | 1,000 | 0.23 |
| 2:1 | 4,000 | 1,999 | 0.33 |
| 1:1 | 4,000 | 4,002 | 0.53 |
| 1:1 | 2,000 | 1,999 | 0.27 |
| 1:2 | 2,000 | 4,002 | 0.47 |
| 1:3 | 1,000 | 3,001 | 0.33 |
| 1:4 | 1,000 | 4,002 | 0.43 |
| 1:5 | 1,000 | 5,000 | 0.53 |
| 1:5 | 500 | 2,499 | 0.27 |
| 1:6 | 500 | 3,001 | 0.32 |
| 1:8 | 500 | 4,002 | 0.42 |
| 1:10 | 500 | 5,000 | 0.52 |
| | 0 | 9,996 | 1.00 |

TABLE 2

| Ratio | Lactic Acid, ppm | Caprylic Acid, ppm | SI |
|---|---|---|---|
| | 6,000 | 0 | 1.00 |
| 2000:1 | 4,000 | 2 | 0.70 |
| 1000:1 | 4,000 | 4 | 0.73 |
| 800:1 | 4,000 | 5 | 0.75 |
| 500:1 | 4,000 | 8 | 0.80 |
| 400:1 | 4,000 | 10 | 0.83 |
| 500:1 | 2,000 | 4 | 0.40 |
| 400:1 | 2,000 | 5 | 0.42 |
| 250:1 | 2,000 | 8 | 0.47 |
| 200:1 | 2,000 | 10 | 0.50 |
| 100:1 | 2,000 | 20 | 0.67 |
| 200:1 | 1,000 | 5 | 0.25 |
| 125:1 | 1,000 | 8 | 0.30 |
| 100:1 | 1,000 | 10 | 0.33 |
| 50:1 | 1,000 | 20 | 0.50 |
| 25:1 | 500 | 20 | 0.42 |
| 5:1 | 250 | 50 | 0.88 |
| | 0 | 60 | 1.00 |

The synergistic ratios of lactic acid/caprylic acid range from 2000:1 to 1:10. The lactic acid/caprylic acid combinations show enhanced control of fungi (yeast and mold).

1. A synergistic microbicidal composition comprising of lactic acid and caprylic acid.

2. The synergistic microbicidal composition of claim 1 wherein the ratio of lactic acid to caprylic acid is 2000:1 to 1:10.

3. A method of using the synergistic microbial composition of claim 1 comprising adding the composition of claim 1 into a cosmetic, toiletry or household, industrial and institutional product.

4. A cosmetic, toiletry or household, industrial and institutional product containing the synergistic microbicidal composition of claim 1
